(19) United States
(12) Patent Application Publication
(10) Pub. No.: US 2023/0392206 A1
(43) Pub. Date: Dec. 7, 2023

(54) METHODS OF TREATMENT AND DIAGNOSIS OF PARKINSON'S DISEASE ASSOCIATED WITH WILD-TYPE LRRK2

(71) Applicant: NEURON23, INC., South San Francisco, CA (US)

(72) Inventors: Mike Nalls, South San Francisco, CA (US); Peter Heutink, San Francisco, CA (US); Adam Knight, San Francisco, CA (US)

(21) Appl. No.: 18/032,073

(22) PCT Filed: Oct. 25, 2021
(86) PCT No.: PCT/US2021/056443

§ 371 Date: Apr. 14, 2023

Related U.S. Application Data
(60) Provisional application No. 63/105,645, filed on Oct. 26, 2020.

(51) Int. Cl. C12Q 1/6883; A61K 31/415; A61K 31/437; A61K 31/519; A61K 31/551

(52) U.S. Cl. CPC C12Q 1/6883; A61K 31/415; A61K 31/437; A61K 31/519; A61K 31/551; C12Q 2600/156; C12Q 2600/106; C12Q 2600/112

(57) ABSTRACT

The invention provides methods of treating patients with Parkinson's disease (PD) associated with wild-type LRRK2. The invention recognizes that analysis of genetic modifiers of LRRK2 in such patients allows identification of those patients who will respond to LRRK2 inhibitors. Thus, the invention provides methods of identifying PD patients who will respond to LRRK2 inhibitors and methods of treating such patients.

### FIELD OF THE INVENTION

The invention relates to methods of treating and diagnosing patients with Parkinson's disease associated with wild-type LRRK2.

### BACKGROUND

Parkinson's disease (PD) is a progressive neurodegenerative disease that affects over six million people globally. PD is usually recognized initially by motor impairment, with the cardinal symptoms being tremor, rigidity, slowness of movement, and difficulty with walking. In later stages, PD also produces neuropsychiatric disorders, including dementia, depression, and anxiety. PD afflicts more than 1% of people over the age of 60 and results in more than 100,000 deaths per year.

PD is thought to result from a confluence of genetic and environmental factors. Numerous mutations associated with familial PD have been identified, but 85-90% of PD cases are idiopathic. In PD cases that can be linked to known genetic factors, mutations in the LRRK2 gene are the most common cause of both familial and idiopathic PD. LRRK2 encodes a protein kinase that is expressed in multiple tissues including regions of the brain associated with PD such as the basal ganglia, and disease-causing mutations result in enhanced kinase activity. However, recent evidence indicates that some cases of PD are associated with increased activity of wild-type, i.e., non-mutant, LRRK2.

Because no cure for PD exists, current treatments focus on alleviating symptoms, particularly motor impairment. The predominant approach for decades has been to enhance dopaminergic function using the dopamine precursor levodopa, a dopamine agonist, or a monoamine oxidase inhibitor. However, such medications lose their effectiveness as the disease progresses, and eventually their side effects outweigh their benefits. More recently, the use of LRRK2 inhibitors has been investigated for treatment of PD cases associated with mutant forms of the LRRK2 kinase. In the vast majority of PD cases, however, no mutation in LRRK2 can be identified. Unfortunately, for PD patients with wild-type LRRK2, there is no way to identify the subset of patients whose disease is associated with elevated LRRK2 activity, and LRRK2 inhibitors cannot be given to PD patients indiscriminately due to the risk of harm to patients who do not have pathological LRRK2 activity. Consequently, existing treatments for most PD patients are inadequate, and millions of people continue to suffer from the progressive and debilitating effects of the disease.

### SUMMARY

The invention provides methods of determining whether a PD patient with wild-type LRRK2 will be more likely to respond to a LRRK2 inhibitor using genetic modifiers of LRRK2 in the patient's genome as indicators. The invention recognizes that genetic modifiers of LRRK2 may cause changes, e.g., increases or decreases, in the level or activity of the LRRK2 kinase or may otherwise alter LRRK2 signaling pathways via upstream or downstream regulators and thus contribute to PD etiology. Consequently, PD patients who have one or more such modifiers may benefit from pharmacotherapy using a LRRK2 inhibitor despite having LRRK2 alleles that produce normal forms of the kinase. Thus, genetic modifiers of LRRK2 activity serve as indicators to determine whether LRRK2 inhibitor therapy is appropriate for a given individual. Methods of the invention are useful both for identifying PD patients as candidates for LRRK2 inhibitor therapy and for treating such patients.

In an aspect, the invention provides methods of treating a subject having Parkinson's disease associated with wild-type LRRK2 by providing a LRRK2 inhibitor to a subject that presents with Parkinson's disease and that has wild-type LRRK2 and a genetic modifier of wild type LRRK2 such that the subject will respond to the LRRK2 inhibitor, thereby treating Parkinson's disease associated with wild-type LRRK2 in the subject.

The genetic data may comprise any type of data on the composition and/or expression of one or more genes in the subject. The genetic data may include one or more of exomic, genomic, genotypic, proteomic, sequence, and transcriptomic data.

The genetic modifier may be any genetic element that modifies, or correlates with the change in activity of, LRRK2 expression or activity, or that causes a change in protein levels associated with disease burden (whether increased or reduced). The genetic modifier may increase or decrease expression and/or activity of LRRK2; the genetic modifier may also increase or reduce degradation of LRRK2. The genetic modifier may be an amplification, deletion, duplication, fusion, insertion, inversion, rearrangement, single nucleotide polymorphism (SNP), substitution, or translocation. The genetic modifier may lie within a coding region or a non-coding region in the subject's genome. The genetic modifier may be associated with family history and genetically ascertained Ashkenazi status.

The SNP may be rs10784722, rs10877877, rs10879122, rs11181542, rs113111234, rs113736300, rs12230765, rs12816484, rs12829831, rs13377670, rs141551396, rs144377852, rs149173058, rs17580794, rs17621741, rs1838354, rs184120094, rs188535877, rs188583486, rs188604552, rs189517205, rs200611801, rs200907772, rs201889643, rs201944175, rs2406426, rs2406860, rs285561, rs34566033, rs368141132, rs369084695, rs371700002, rs371905892, rs373439540, rs376468815, rs377104202, rs377627337, rs384234, rs61920964, rs6581941, rs6650226, rs71078241, rs7304080, rs73088926, rs74434364, rs74842215, rs75043969, rs78468120, rs7960429, rs7979420, rs76904798, rs57025360, rs112515153, rs10877877, rs10784722, rs4272849, rs2404832, rs117534366, rs1838343, rs10880342, rs11177660, rs183028452, rs116912628, rs147755361, rs11584630, rs3793397, rs111794893, rs4931640, rs526507, rs79307177, rs187116363, rs71609573, rs74390551, rs144665441, rs1718880, rs1991401, rs11052225, rs145801597, rs72907976, rs147286120, rs378690, rs73188365, rs610037, rs75479531, rs1112191556, rs308303, rs10790282, rs3729912, rs4326638, rs4414548, rs13009437, rs56045011, rs6858566, rs4425, rs11052253, or any other SNPs in linkage disequilibrium (LD) with these SNPs that would be suitable as a proxy for these SNPs.

The LRRK2 inhibitor may be CZC-25146, CZC-54252, DNL151, DNL201, GNE-7915, GNE-0877, GSK2578215A, HG-10-102-01, JH-II-127, K252A, K252B, LRRK2-IN-1, MLi-2, PF-06447475, or staurosporine.

The LRRK2 inhibitor may be a compound of one of formulas (I), (II), (III), or (IV):

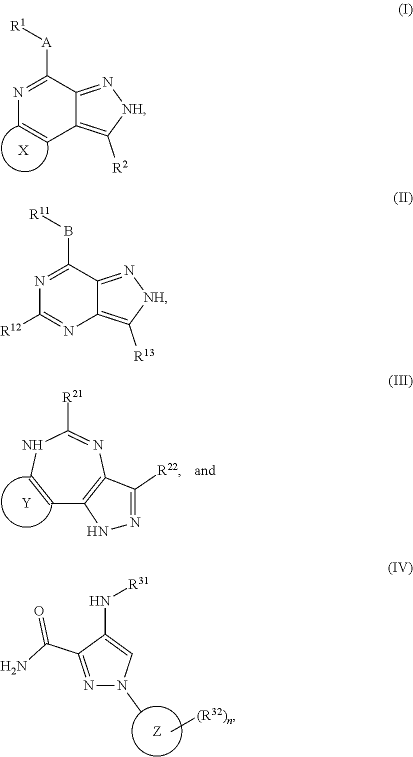

wherein:
A is NH, O, S, C═O, NR³ or CR⁴R⁵;
X is an optionally substituted arylene, heteroarylene, cycloalkylene, heterocycloalkylene, alkylcycloalkylene, heteroalkylcycloalkylene, aralkylene or heteroaralkylene group;
R¹ is an optionally substituted alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R² is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R⁴ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group; and
R⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
B is NH, O, S, C═O, NR¹⁴ or CR¹¹R¹⁶;
R¹¹ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹² is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group, wherein R¹² is bound to the pyrimidine ring of formula (II) via a carbon-carbon bond;
R¹³ is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁴ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R¹⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁶ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R²¹ is aryl or heteroaryl, each of which is optionally substituted;
R²² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl; and
Y is aryl or 5- or 6-membered heteroaryl;
   wherein each of the C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, and heteroaryl is optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;
R³¹ is C(O)CH₂R³³, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R³² is independently halo, haloalkyl, optionally substituted alkoxyl, optionally substituted alkyl, optionally substituted heteroalkyl, optionally substituted alkenyl, optionally substituted heteroalkenyl;
R³³ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
Z is cycloalkyl, cycloheteroalkyl, cycloalkenyl, cycloheteroalkenyl, aryl, or heteroaryl; and
n is 0-5,
   or a pharmaceutically acceptable salt of any compound described above.

In another aspect, the invention provides methods of determining whether a subject having Parkinson's disease associated with wild-type LRRK2 will respond to a LRRK2 inhibitor. The methods includes conducting an assay on a sample from a subject that has Parkinson's disease associated with wild-type LRRK2 in order to obtain genetic data from a subject, generating a report that identifies one or more genetic modifier of LRRK2 in the genetic data, wherein the one or more genetic modifiers in the LRRK2 network are indicative that the subject having Parkinson's disease associated with wild-type LRRK2 will be responsive to a LRRK2 inhibitor, and providing the report to a physician such that the physician prescribe or provide the subject with a LRRK2 inhibitor.

The genetic data may be any type of genetic data described above.

The genetic modifier may be any type of genetic modifier of LRRK2 described above. The genetic modifier may be any of the SNPs listed above.

The LRRK2 inhibitor may be any of those described above.

In another aspect, the invention provides methods of treating a subject having Parkinson's disease associated with wild-type LRRK2. The methods include receiving genetic data that identifies one or more genetic modifier of LRRK2, wherein the one or more genetic modifiers are indicative that a subject having Parkinson's disease associated with wild-type LRRK2 will be responsive to a LRRK2 inhibitor, and prescribing or providing the subject with a LRRK2 inhibitor.

The genetic data may be any type of genetic data described above.

The genetic modifier may be any type of genetic modifier of LRRK2 described above. The genetic modifier may be any of the SNPs listed above.

The LRRK2 inhibitor may be any of those described above.

In another aspect, the invention provides LRRK2 inhibitors for use in treatment of PD associated with wild-type LRRK2.

The subject may have one or more genetic modifiers of LRRK2, such as any of those described above.

The use may include receiving or obtaining genetic data, such as any of the genetic data described above.

The LRRK2 inhibitor may be any of those described above.

### DETAILED DESCRIPTION

Parkinson's disease (PD) is a progressive neurodegenerative disease that is caused by both genetic and environmental factors. One gene that plays a role in the development of some cases of PD is LRRK2, which encodes kinase that is expressed in multiple tissues including regions of the brain associated with PD such as the basal ganglia. Mutations in LRRK2 are the most common known genetic cause of PD, but patients with LRRK2 mutations make up a small fraction of the total number of PD cases. Nonetheless, the pathology in some patients with wild-type, i.e., non-mutant, LRRK2, appears to resemble that in patients with mutant LRRK2. In particular, disease-causing mutations in LRRK2 result in increased activity of the LRRK2 kinase, and it has recently been shown that LRRK2 activity is elevated in some PD patients with wild-type LRRK2.

Various inhibitors of LRRK2 are currently being investigated as PD therapeutics. Such drugs hold promise for PD patients with LRRK2 mutations. However, the use of LRRK2 inhibitors to treat PD patients with wild-type LRRK2 is problematic due to the varied etiology of the disease. Although patients with enhanced activity of wild-type LRRK2 would benefit from LRRK2 inhibitors, inhibition of LRRK2 may not be effective in PD patients who have normal levels of LRRK2 activity and whose disease pathology is attributable to changes in other molecular pathways. Because the neurons that express LRRK2 are located in the mid-brain and extremely difficult to access, activity of the kinase cannot be evaluated in living patients. Consequently, to date there has not been a means for identifying the subset of PD patients who have wild-type LRRK2 but could still benefit from LRRK2 inhibition.

The invention solves this problem by using genetic modifiers of LRRK2 activity to determine whether a PD patient with wild-type LRRK2 will likely benefit from a LRRK2 inhibitor. The invention recognizes that genetic variations outside of the LRRK2 locus affect the expression or activity of the LRRK2 kinase, and the presence of certain genetic markers correlates with changes, e.g., increase or decrease, in LRRK2 expression or activity. Consequently, methods of the invention allow candidates for LRRK2 pharmacotherapy to be identified based on genetic data that can be easily obtained from the patient. Thus, for a subset of PD patients, the invention unlocks the therapeutic potential of a class of drugs that were previously not recommended for them.

### Parkinson's Disease and Treatments Thereof

Parkinson's disease (PD) is a progressive neurodegenerative disease of the central nervous system. In early stages, the disease affects the motor system, and the cardinal symptoms are tremor, rigidity, slowness of movement, and difficulty with walking. Cognitive and behavioral symptoms, such as dementia, depression, and anxiety, often appear in later stages of PD. PD usually occurs in people over the age of 60, of whom about 1% are affected, but so-called early-onset PD may occur before the age of 50.

PD is characterized by the death of cells in the basal ganglia, including dopamine-secreting neurons, astrocytes, and microglia of the substantia nigra. Five mechanisms for neuronal death in PD have been proposed. First, the oligomerization of proteins, such as alpha-synuclein, into aggregates called Lewy bodies may lead directly to cell death. A second proposed cause is the dysregulation of autophagy, particularly degradation of mitochondria. Another proposed mechanism is that mitochondrial dysfunction leads to decreased energy production and an increase in reactive oxygen species. A fourth proposed mechanism is that due to neuroinflammation as a result of secretion of pro-inflammatory factors by the microglia. Finally, it has been proposed that breakdown of the blood-brain barrier allows plasma proteins to leak into the substantia nigra and promote apoptosis.

It is thought that PD results from a combination of genetic and environmental factors. In some cases, genetic mutations that increase the risk of PD are heritable, and about 10-15% of individuals with PD have a first-degree relative who has the disease. However, most instances of PD are idiopathic or “sporadic.” Genes with mutations that have been implicated in PD include CHCHD2, DJ1/PARK7, DNAJC13, EIF4G1, GBA, LRRK2/PARK8, PINK1, PRKN, SNCA, UCHL1, and VPS35. For both familial and sporadic PD, the most common known cause is mutation of LRRK2. Disease-causing mutations in LRRK2 result in a form of the kinase that has increased activity. Enhanced activity of wild-type LRRK2 activity has recently been implicated in idiopathic PD as well. The role of LRRK2 in PD is described in, for example, Chen, et al., Leucine-Rich Repeat Kinase 2 in Parkinson's Disease: Updated from Pathogenesis to Potential Therapeutic Target, Eur Neurol. 2018; 79 (5-6):256-265, doi: 10.1159/000488938. Epub 2018 Apr. 27; Di Maio, et al., LRRK2 activation in idiopathic Parkinson's disease, Sci Transl Med. 2018 Jul. 25; 10(451):eaar5429, doi: 10.1126/scitranslmed.aar5429; Taymans and Greggio, LRRK2 Kinase Inhibition as a Therapeutic Strategy for Parkinson's Disease, Where Do We Stand? Curr Neuropharmacol. 2016; 14(3):214-25, doi: 10.2174/1570159x13666151030102847, the contents of each of which are incorporated herein by reference.

Several behavioral and environmental conditions are known to increase the risk of developing PD. Risk factors associated with PD include exposure to pesticides and a history of head injury. Caffeine consumption and tobacco use are associated with decreased risk of PD. Low concentration of urate in the blood is associated with an increased risk of PD.

Management of PD usually entails pharmacological stimulation of the dopaminergic system. The most widely-used drug for treatment of PD is levodopa, which is enzymatically converted to dopamine in dopaminergic neurons. Dopamine agonists, such as bromocriptine, pergolide, pramipexole, ropinirole, piribedil, cabergoline, apomorphine, and lisuride, may also be used to treat PD. A third class of drugs for treatment of PD includes inhibitors of monoamine oxidase, such as selegiline and rasagiline.

Identification of Genetic Modifiers from Genetic Data

The invention recognizes that genetic modifiers of LRRK2 serve as indicators that PD patients having wild-type LRRK2 are likely to benefit from pharmacotherapy using one or more LRRK2 inhibitors. A genetic modifier of LRRK2 may be one or more genetic elements (e.g., a single genetic element alone or any combination(s) of genetic elements) that operably modifies LRRK2 (e.g., wild-type LRRK2), e.g., that alters the expression, degradation, localization (e.g., within a cell or across cell types), binding, or activity of LRRK2, including the LRRK2 gene, transcripts of the LRRK2 gene, and polypeptide products of the LRRK2 gene, in a subject. For example and without limitation, a genetic modifier may alter, e.g., increase or decrease, expression, activity, stability, binding, localization, degradation, transcription, or translation of LRRK2, including the LRRK2 gene, transcripts of the LRRK2 gene, and polypeptide products of the LRRK2 gene. In certain embodiments, a genetic modifier of LRRK2 may be a structural variation in the genome of the subject. For example and without limitation, a genetic modifier may be an amplification, deletion, duplication, fusion, insertion, inversion, rearrangement, single nucleotide polymorphism (SNP), substitution, or translocation. SNPs that may be genetic modifiers of LRRK2 are listed in Example 1. In addition, any other SNPs that are in linkage disequilibrium (LD) with the SNPs listed in Example 1 may be used as a genetic modifier. A genetic modifier may be a cis-regulatory element, such as a promoter, enhancer, silencer, or operator. The cis-regulatory element may regulate the binding of one or more proteins to DNA in proximity to LRRK2. The cis-regulatory element may affect binding of a histone, transcription factor, initiation factor, helicase, polymerase, or component of any of the aforementioned proteins. A genetic modifier may be a trans-acting factor. The trans-acting factor may affect transcription or translation of LRRK2. A genetic modifier may be in any region of the subject's genome. A genetic modifier may lie within a coding region or non-coding region of the subject's genome. The coding region may be in LRRK2 or in another gene. A genetic modifier may lie within the LRRK2 coding region but not alter the sequence of the LRRK2 polypeptide, the size of the LRRK2 polypeptide, or both.

Methods of the invention may include identification or analysis of one or more genetic modifiers of LRRK2 in genetic data obtained from a subject. The genetic data may comprise any type of data on the composition and/or expression of one or more genes in the subject. The genetic data may include one or more of exomic, genomic, genotypic, proteomic, sequence, and transcriptomic data. The genetic data may include data on one or more genes known to be associated with PD, such as any of those described above.

Genetic modifiers may be identified from genetic data using any suitable method. In some embodiments, the genetic data collected from the subject is compared to a reference set of data in order to provide a probability of responsiveness to a LRRK2 inhibitor. The reference set may include data collected from individuals that do not have PD. Phenotypic data from subjects and reference individuals may also be used. Phenotypic data may contain traits associated with PD, including PD symptoms or PD risk factors, such as those described above. Data may include outcomes, such as whether the individual responded to LRRK2 inhibitor treatment.

The invention provides methods and systems for predicting a subject's responsiveness to a LRRK2 inhibitor based on the subject's phenotypic traits and/or genotypic data. In some embodiments, methods and systems of the invention use a diagnostic signature for predicting responsiveness. The diagnostic predictor can be based on any appropriate pattern recognition method that receives input data representative of a plurality of responsiveness-associated phenotypic traits, such as molecular signatures of (1) LRRK2-like manifestations of PD observed in carriers of LRRK2 deleterious variants, (2) PD of apparently unknown mechanism, and (3) appropriate controls, and provides an output that indicates a probability that the subject will respond to a LRRK2 inhibitor. The diagnostic predictor may be trained with data from a plurality of individuals for whom phenotypic traits, medical interventions, and LRRK2 inhibitor response outcomes are known. The plurality of individuals used to train the diagnostic predictor is also known as the training population. For each individual in the training population, the training data comprises (a) data representative of a plurality of phenotypic traits; (b) medical interventions; and (c) LRRK2 inhibitor response information. LRRK2 inhibitor response outcome may not be required to generate a diagnostic signature. LRRK2 inhibitor responses can be evaluated in a prospectively selected patient population. Various diagnostic predictors that can be used in conjunction with the present invention are described below. In some embodiments, additional individuals having known trait profiles and LRRK2 response outcomes can be used to test the accuracy of the diagnostic predictor obtained using the training population. Such additional patients are known as the testing population.

In certain embodiments, the methods of invention use a diagnostic predictor, also called a classifier, for determining the probability of responding to LRRK2 inhibition. As noted above, the diagnostic predictor can be based on any appropriate pattern recognition method that receives a profile, such as a profile based on a plurality of phenotypic traits and provides an output comprising data indicating a that a patient is more or less likely to respond to a LRRK2 inhibitor, and may include possible risks and benefits of treatment with such an inhibitor. The profile can be obtained by completion of a questionnaire containing questions regarding certain phenotypic traits or the collection of a biological sample to obtain genotypic data or a combination thereof. The diagnostic predictor is trained with training data from a training population of individuals for whom phenotypic traits, medical interventions, and LRRK2 inhibitor response outcomes are known.

A diagnostic predictor based on any of such methods can be constructed using the profiles and diagnostic data of the training patients. Such a diagnostic predictor can then be used to predict the LRRK2 inhibitor response of a subject based on her profile of phenotypic traits, genotypic traits, or both. The methods can also be used to identify traits that discriminate between responding and not responding to LRRK2 inhibition using a trait profile and diagnostic data of the training population.

In one embodiment, the diagnostic predictor can be prepared by (a) generating a reference set of individuals for whom phenotypic traits, medical interventions, and LRRK2 response outcomes are known; (b) determining for each trait, a metric of correlation between the trait and LRRK2 response outcome in a plurality of individuals having known LRRK2 response outcomes at a predetermined time; (c) selecting one or more traits based on said level of association; (d) training a diagnostic predictor, in which the diagnostic predictor receives data representative of the traits selected in the prior step and provides an output indicating a probability of responding to LRRK2 inhibition, with training data from the reference set of subjects including assessments of traits taken from the individuals.

Various known statistical pattern recognition methods can be used in conjunction with the present invention. Suitable statistical methods include, without limitation, logic regression, ordinal logistic regression, linear or quadratic discriminant analysis, clustering, principal component analysis, nearest neighbor classifier analysis, and Cox proportional hazards regression. Non-limiting examples of implementing particular diagnostic predictors in conjunction are provided herein to demonstrate the implementation of statistical methods in conjunction with the training set.

In some embodiments, the diagnostic predictor is based on a regression model, preferably a logistic regression model. Such a regression model includes a coefficient for each of the markers in a selected set of markers of the invention. In such embodiments, the coefficients for the regression model are computed using, for example, a maximum likelihood approach. Cox proportional hazards regression also includes a coefficient for each of the markers in a selected set of markers of the invention. Cox proportional hazards regression incorporates censored data (individuals in the reference set that did not return for treatment). In such embodiments, the coefficients for the regression model are computed using, for example, a maximum partial likelihood approach.

Some embodiments of the present invention provide generalizations of the logistic regression model that handle multicategory (polychotomous) responses. Such embodiments can be used to discriminate an organism into one or three or more diagnosis groups. Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference. Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the selected phenotypic traits serve as the requisite continuous independent variables. The diagnosis group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how a selected phenotypic trait manifests in the two groups (e.g., a group that responds to LRRK2 inhibition and a group that does not) and how the selected trait correlates with the manifestation of other traits. For example, LDA can be applied to the data matrix of the N members in the training sample by K genes in a combination of genes described in the present invention. Then, the linear discriminant of each member of the training population is plotted. Ideally, those members of the training population representing a first subgroup (e.g., those subjects that do not respond to LRRK2 inhibition) will cluster into one range of linear discriminant values (e.g., negative) and those member of the training population representing a second subgroup (e.g., those subjects that respond to LRRK2 inhibition) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

In some embodiments of the present invention, decision trees are used to classify patients using expression data for a selected set of molecular markers of the invention. Decision tree algorithms belong to the class of supervised learning algorithms. The aim of a decision tree is to induce a classifier (a tree) from real-world example data. This tree can be used to classify unseen examples which have not been used to derive the decision tree.

A decision tree is derived from training data. An example contains values for the different attributes and what class the example belongs. In one embodiment, the training data is data representative of a plurality of phenotypic traits, medical interventions, and LRRK2 inhibition response outcomes.

The following algorithm describes a decision tree derivation:

Tree(Examples, Class, Attributes)
Create a root node
If all Examples have the same Class value, give the root this label
Else if Attributes is empty label the root according to the most
common value
Else begin
Calculate the information gain for each attribute
Select the attribute A with highest information gain and make
this the root attribute
For each possible value, v, of this attribute
Add a new branch below the root, corresponding to A = v
Let Examples(v) be those examples with A = v
If Examples(v) is empty, make the new branch a leaf node labeled
with the most common value among Examples
Else let the new branch be the tree created by
Tree(Examples(v), Class, Attributes-{A})
end

A more detailed description of the calculation of information gain is shown in the following. If the possible classes vi of the examples have probabilities P(vi) then the information content I of the actual answer is given by:

I(P(v₁), . . . ,P(vₙ))=nΣi=1−P(vᵢ)log₂ P(vᵢ)

The I-value shows how much information we need in order to be able to describe the outcome of a classification for the specific dataset used. Supposing that the dataset contains p positive examples (e.g., responders) and n negative examples (e.g., non-responders), the information contained in a correct answer is:

I(p/p+n,n/p+n)=−p/p+n log₂p/p+n−n/p+n log₂n/p+n

where log 2 is the logarithm using base two. By testing single attributes the amount of information needed to make a correct classification can be reduced. The remainder for a specific attribute A (e.g., a trait) shows how much the information that is needed can be reduced.

Remainder(A)=vΣi=1pᵢ+nᵢ/p+nI(pᵢ/pᵢ+nᵢ,nᵢ/pᵢ+nᵢ)

“v” is the number of unique attribute values for attribute A in a certain dataset, “i” is a certain attribute value, “pᵢ” is the number of examples for attribute A where the classification is positive (e.g., responder), “nᵢ” is the number of examples for attribute A where the classification is negative (e.g., non-responder).

The information gain of a specific attribute A is calculated as the difference between the information content for the classes and the remainder of attribute A:

Gain(A)=I(p/p+n,n/p+n)−Remainder(A)

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when an exemplary embodiment of a decision tree is used, the data representative of a plurality of phenotypic traits across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of traits are used to construct the decision tree. Then, the ability for the decision tree to correctly classify members in the test set is determined. In some embodiments, this computation is performed several times for a given combination of molecular markers. In each iteration of the computation, the members of the training population are randomly assigned to the training set and the test set. Then, the quality of the combination of traits is taken as the average of each such iteration of the decision tree computation.

In some embodiments, the phenotypic traits and/or genotypic data are used to cluster a training set. For example, consider the case in which ten genes described in the present invention are used. Each member m of the training population will have expression values for each of the ten genes. Such values from a member m in the training population define the vector:

X₁ₘX₂ₘX₃ₘX₄ₘX₅ₘX₆ₘX₇ₘX₈ₘX₉ₘX₁₀ₘ

where Xᵢₘ is the expression level of the i^{th} gene in organism m. If there are m organisms in the training set, selection of i genes will define m vectors. Note that the methods of the present invention do not require that each the expression value of every single trait used in the vectors be represented in every single vector m. In other words, data from a subject in which one of the i^{th} traits is not found can still be used for clustering. In such instances, the missing expression value is assigned either a “zero” or some other normalized value. In some embodiments, prior to clustering, the trait expression values are normalized to have a mean value of zero and unit variance.

Those members of the training population that exhibit similar expression patterns across the training group will tend to cluster together. A particular combination of traits of the present invention is considered to be a good classifier in this aspect of the invention when the vectors cluster into the trait groups found in the training population. For instance, if the training population includes patients with good or poor prognosis, a clustering classifier will cluster the population into two groups, with each group uniquely representing either good or poor prognosis.

Clustering is described on pages 211-256 of Duda and Hart, Pattern Classification and Scene Analysis, 1973, John Wiley & Sons, Inc., New York. As described in Section 6.7 of Duda, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda.

Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda. Criterion functions are discussed in Section 6.8 of Duda.

More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Nearest neighbor classifiers are memory-based and require no model to be fit. Given a query point x₀, the k training points x(r), r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎>−xₒ∥.

Typically, when the nearest neighbor algorithm is used, the expression data used to compute the linear discriminant is standardized to have mean zero and variance 1. In the present invention, the members of the training population are randomly divided into a training set and a test set. For example, in one embodiment, two thirds of the members of the training population are placed in the training set and one third of the members of the training population are placed in the test set. Profiles represent the feature space into which members of the test set are plotted. Next, the ability of the training set to correctly characterize the members of the test set is computed. In some embodiments, nearest neighbor computation is performed several times for a given combination of phenotypic traits. In each iteration of the computation, the members of the training population are randomly assigned to the training set and the test set. Then, the quality of the combination of traits is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York.

The pattern classification and statistical techniques described above are merely examples of the types of models that can be used to construct a model for classification. It is to be understood that any statistical method can be used in accordance with the invention. Moreover, combinations of these described above also can be used. Further detail on other statistical methods and their implementation are described in U.S. Pat. No. 10,181,009, incorporated by reference herein in its entirety.

It is understood that during the course of treatments, individuals that make-up the reference set may drop out prior to determining their LRRK2 inhibition response. It is not known whether those individuals eventually respond to LRRK2 inhibition. Simply omitting those individuals from the reference set would bias the reference data set by omitting characteristics of individuals having a poor prognosis for responding. Such a bias would result in reporting an overly optimistic probability of responding to treatment with LRRK2 inhibitors.

With systems and methods of the invention, rather than omitting those subjects wholesale, the present invention takes advantage of certain methods of statistical analysis to account for dropouts. The Kaplan-Meier method, for example, can be used to censor or exclude data for individuals in the reference set that did not return for treatment. Other forms of statistical analysis can be used in accordance with the present invention to compile the data of the reference set. For example, logistic regression, ordinal logistic regression, Cox proportional hazards regression, and other methods can all be used to compile the data within the reference set. In addition, it is contemplated that the reference set can censor or account for dropouts based on the traits of the individuals rather than making blanket assumptions regarding the responsiveness of the dropouts. For example, rather than simply assuming that a dropout had the same chance of responding as the individuals who continued treatment, or assuming that a dropout had no chance of responding, the present invention can evaluate the traits of the dropouts and informatively censor the dropouts based on such information. In this manner, overly-optimistic estimates (resulting from the assumption that all dropouts had equal chances of responding) or overly-conservative estimates (resulting from the assumption that the dropouts had no chances of responding) are avoided.

In certain aspects, the present invention incorporates the use of artificial censoring to account for dropouts. In artificial censoring, participants are censored when they meet a predefined study criterion, such as exposure to an intervention, noncompliance with their treatment regimen, or the occurrence of a competing outcome. Further analytical methods, such as inverse-probability-of-censoring weights (IPCW), can then be used to determine what the survival experiences of the artificially censored participants would have been had they never been exposed to the intervention, complied, or not developed the competing outcome. In some embodiments, methods encompassing the use of artificial censoring and further, the use of IPCW are encompassed by the invention to account for dropouts in the reference set. Additional detail regarding the use of artificial censoring and the use of IPCW is described in Howe et al., Limitation of inverse probability-of-censoring weights in estimating survival in the presence of strong selection bias, Am J Epidemiology, 2011, incorporated by reference herein in its entirety.

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random-access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

Methods of the invention may utilize a machine learning system. For example, the machine learning system may learn in a supervised manner, an unsupervised manner, a semi-supervised manner, or through reinforcement learning.

In an unsupervised model or autonomous model, the machine learning system is only given input training data without paired output data from which to identify patterns autonomously. Unsupervised models identify underlying patterns or structures in training data to make predictions for test data. Unsupervised models are advantageous for clustering data, detecting anomalies, and for independently discovering rules for data. The accuracy of unsupervised models is harder to evaluate because there is no predefined output variable to which the system is optimizing. Autonomous models may employ periods of both supervised and unsupervised learning in order to optimize predictions. Unsupervised models are advantageous for training a machine learning system to cluster data into clusters when labeled training data is unavailable. Unsupervised models may use Principal Component Analysis (PCA), Uniform Manifold Approximation and Projection (UMAP). Discriminant analysis may also be used when groups in the training and test data are already known. Discriminant analysis may include linear discriminant analysis (LDA) and quadratic discriminant analysis (QDA)).

In semi-supervised models, the machine learning system is given training data comprising input variables, with output variable pairs available for only a limited pool of the input variables. The model uses the input variables with output variable pairs and the remaining input training data to learn patterns and make inferences in order to generate a prediction on previously unseen test data. A semi-supervised model may advantageously query the user for additional paired output data based on unpaired data. Semi-supervised models are advantageous for training a machine learning system when only an incomplete training data set is available.

In a reinforcement learning model, the machine learning system is given neither input variables nor output variables. Rather, the model provides a “reward” condition and then seeks to maximize the cumulative reward condition by trial and error. A reinforcement learning model is a Markov Decision Process. Supervised, unsupervised, semi-supervised, and reinforcement models are described in Jordan and Mirchell, 2015, Machine learning, Trends, perspectives, and prospects, Science 349(6245):255-260, incorporated by reference.

An example of a supervised learning model is a “decision tree.” Decision trees are non-parametric supervised learning models that use simple decision rules to infer a classification for test data from the features in the test data. In classification trees, test data take a finite set of discrete values, or classes, whereas in regression trees, the test data can take continuous values, such as real numbers. Decision trees have some advantages in that they are simple to understand and can be visualized as a tree starting at the root (usually a single node) and repeatedly branch to the leaves (multiple nodes) that are associated with the classification. See Criminisi, 2012, Decision Forests: A unified framework for classification, regression, density estimation, manifold learning and semi-supervised learning, Foundations and Trends in Computer Graphics and Vision 7(2-3):81-227, incorporated by reference.

Another supervised learning model is a “support-vector machine” (SVM), “support-vector network” (SVN), or support vector classifier (SVC), which are supervised learning models for classification and regression problems. When used for classification of new data into one of two categories, an SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines. Numerical Recipes: The Art of Scientific Computing (3rd ed.). New York: Cambridge University (2007), incorporated herein by reference. Where output variable pairs are unavailable for input variables in the training data, SVMs can be designed as unsupervised or semi-supervised learning models using support vector clustering. See Ben-Hur, 2001, Support Vector Clustering, J Mach Learning Res 2:125-137, incorporated by reference. SVM models can be advantageous for the machine learning system where test data falls into a limited number of possible categories. Additionally, SVM models can be advantageous where only a limited set of training data is available for the machine learning system.

Logistic regression analysis is another statistical process that can be used by the machine learning system to find patterns in training and test data to make predictions. It includes techniques for modeling and analyzing relationships between multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning. Regression models also provide the advantage of being effectively implemented by a variety tools and the model can be easily updated to identify new particles.

SVM systems and logistic regression systems may use a stochastic gradient descent (SGD) approach to fit data. SGDs are advantageous in optimizing the machine learning system utilizing the approach.

Bayesian algorithms may also be used to find patterns in training and test data to make predictions. Bayesian networks are probabilistic graphical models that represent a set of random variables and their conditional dependencies via directed acyclic graphs (DAGs). The DAGs have nodes that represent random variables that may be observable quantities, latent variables, node unknown parameters or hypotheses. Edges represent conditional dependencies; nodes that are not connected represent variables that are conditionally independent of each other. Each is associated with a probability function that takes, as input, a particular set of values for the node's parent variables and gives (as output) the probability (or probability distribution, if applicable) of the variable represented by the node. Bayesian models provide the advantage of generally requiring less training data than other models.

Some models may rely on clustering training data and test data to find patterns and make predictions. A “k-nearest neighbor” (k-NN) model is a supervised non-parametric learning model for classification and regression problems. A k-nearest neighbor model assumes that similar data exists in close proximity and assigns a category or value to each data point based on the k nearest data points. k-NN models may be advantageous when the data has few outliers and can be defined by homogeneous features. Moreover, k-NN models provide the advantage of continuously learning from test data and do not require a training period before identifying material from training data.

An example of an unsupervised learning model that uses clustering is a “k-means” clustering model. A k-means model looks to find clusters of data in input data and test data. K-means models are advantageous when a defined number of clusters are known to exist in the data and are also advantageous when the test data has few outliers and can be defined homogeneous features. Additional models that cluster training data include, for example, farthest-neighbor, centroid, sum-of-squares, fuzzy k-means, and Jarvis-Patrick clustering. k-means and other unsupervised clustering models are advantageous when training data is unavailable or limited.

Trained machine learning models can become “stable learners.” A stable learner is a model that is less sensitive to perturbation of predictions based on new training data. Stable learners can be advantageous where test data is stable, but can be less advantageous where the system needs to continually improve performance to accurately predict new test data that may be less stable. Accordingly, a stable learning model may be advantageous for use by the machine learning system when the types data that may be introduced are known and are unlikely to change.

Several machine learning system types can be combined into final predictive models known as ensembles. Ensembles can be divided into two types: homogenous ensembles and heterogeneous ensembles. Homogenous ensembles combine multiple machine learning models of the same type. Heterogeneous ensembles combine multiple machine learning models of different types. Ensembles can provide an advantage because they can be more accurate than any of the individual base member models (“members”) in the ensemble. The number of members combined in an ensemble may impact the accuracy of a final prediction. Accordingly, it is advantageous to determine the optimal number of members when designing an ensemble system for use by the machine learning system.

Ensembles used by the machine learning system may combine or aggregate outputs from individual members by using “voting”-type methods for classification systems and “averaging”-type methods for regression systems. In a “majority voting” method, each member makes a prediction as to the test data and the prediction that receives more than half of the votes is the final output for the ensemble. If none of the predictions receives more than half of the votes, it may be determined that the ensemble is unable to make a stable prediction. In a “plurality voting” method, the most voted prediction, even if receiving less than half of the votes, may be considered the final output for the ensemble. In a “weighted voting” method, the votes of more accurate members are multiplied by a weight afforded each member based on its accuracy. In a “simple averaging” method, each member makes a prediction for test data and the average of the outputs is calculated. This method reduces overfit and can be advantageous in creating smoother regression models. In a “weighted averaging” method, the prediction output of each member is multiplied by a weight afforded each member based on its accuracy. Voting methods, averaging methods, and weighted methods can be combined to improve the accuracy of ensembles used by the machine learning system.

Members within an ensemble used by the machine learning system can each be trained independently, or new members can be trained utilizing information from previously trained members. In a “parallel ensemble”, the ensemble seeks to provide greater accuracy than individual members by exploiting the independence between members, for example, by training multiple members simultaneously to identify and aggregate the outputs from members. In “sequential ensemble systems”, the ensemble seeks to provide greater accuracy than individual members by exploiting the dependence between members, for example, by utilizing information from a first member regarding the identification of data to improve the training of a second member for identifying data and weighting outputs from members.

Overall accuracy for ensembles used by the machine learning system may be optimized by using ensemble meta-algorithms, for example, a “bagging” algorithm to reduce variance, a “boosting” algorithm to reduce bias, or a “stacking” algorithm to improve predictions.

Boosting algorithms reduce bias and can be used to improve less accurate, or “weak learning” models. A member may be considered a “weak learning” model if it has a substantial error rate, but its performance is non-random. Boosting algorithms incrementally build the ensemble by training each member sequentially with the same training data set, examining prediction errors for test data, and assigning weights to training data based on the difficulty for members to make an accurate prediction. In each sequential member trained, the algorithm emphasizes training data that previous members found difficult. Members are then weighted based on the accuracy of their prediction outputs in view of the weight applied to the training data. The predictions from each member may be combined by weighted voting-type or weighted averaging-type methods. Boosting algorithms are advantageous when combining multiple weak learning models. Boosting algorithms may, however, result in over-fitting test data to training data. Examples of boosting algorithms include AdaBoost, gradient boosting, eXtreme Gradient Boost (XGBoost). See Freund, 1997, A decision-theoretic generalization of on-line learning and an application to boosting, J Comp Sys Sci 55:119; and Chen, 2016, XGBoost: A Scalable Tree Boosting System, arXiv:1603.02754, both incorporated by reference.

Bagging algorithms or “bootstrap aggregation” algorithms reduce variance by averaging together multiple estimates from members. Bagging algorithms provide each member with a random sub-sample of a full training data set, with each random sub-sample known as a “bootstrap” sample. In the bootstrap samples, some data from the training data set may appear more than once and some data from the training data set may not be present. Because sub-samples can be generated independently from one another, training can be done in parallel. The predictions for test data from each member are then aggregated, such as by voting-type or averaging-type methods.

An example of a bagging algorithm that may be used by the machine learning system is a “random forest”. In a random forest, the ensemble combines multiple randomized decision tree models. Each decision tree model is trained from a bootstrap sample from a training set for test data. The training set itself may be a random subset of features from an even larger training set. By providing a random subset of the larger training set at each split in the learning process, spurious correlations that can result from the presence of individual features that are strong predictors for the output variable are reduced. By averaging predictions for test data, variance of the ensemble decreases resulting in an improved prediction of test data. Random forests may be autonomous models and may include periods of both supervised and unsupervised learning. Bagging may be less advantageous in optimizing an ensemble combining stable learning systems, since stable learning systems tend provide generalized outputs with less variability over the bootstrap samples. Random forests are advantageous for use by the machine learning system to identify data by providing a great degree of versatility in identifying test data and reducing spurious identification by the machine learning system. See Breiman, 2001, Random Forests, Machine Learning 45:5-32, incorporated by reference.

Stacking algorithms or “stacked generalization” algorithms improve predictions by using a meta-machine learning model to combine and build the ensemble. In stacking algorithms, base member models are trained with a training dataset and generate as an output a new dataset. This new dataset is then used as a training dataset for the meta-machine learning model to build the ensemble. Stacking algorithms are generally advantageous for use by the machine learning system to identify test data when building heterogeneous ensembles. Ensembles are described in Villaverde et al., 2019, On the adaptability of ensemble methods for distribution classification systems: A comparative analysis, International Journal of Distributed Sensor Networks 15(7); and Heitor et al., 2017, A Survey of Ensemble Learning for Data Stream Classification, 50(2):Art. 23, each incorporated by reference.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015); each of the aforementioned references are incorporated by reference. The advantage of using a machine learning system based on a neural network architecture is that neural networks are able to learn patterns and correlations by themselves and produce outputs that are not limited by the training data provided to them.

Deep learning neural networks (also known as deep structured learning, hierarchical learning, or deep machine learning) include a class of machine learning operations that may be used by the classifier that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower level features to form a hierarchical representation. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Within a neural network that may be used by the machine learning system, nodes are connected in layers, and signals travel from the input layer to the output layer. Each node in the input layer may correspond to a respective feature from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are advantageously learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights and is sometimes done to train the network using known correct outputs. See WO 2016/182551, U.S. Pub. 2016/0174902, U.S. Pat. No. 8,639,043, and U.S. Pub. 2017/0053398, each incorporated by reference.

Features from test or training data can be represented by a deep learning network in many ways, such as a vector of intensity values per pixel in the image, or in a more abstract way as a set of edges, regions of particular shape, etc. Those features are represented at nodes in the network. Preferably, each feature is structured as numerical feature or vector that represents the image feature. This provides a numerical representation of objects, for example from an image, since such representations facilitate processing and statistical analysis. Numerical features are often combined with weights using a dot product in order to construct a linear predictor function that is used to determine a score for making a prediction.

The vector space associated with those feature vectors may be referred to as the feature space. In order to reduce the dimensionality of the feature space, dimensionality reduction may be employed by networks used by the classifier. Higher-level features can be obtained from already available features and added to the feature vector, in a process referred to as feature construction. Feature construction is the application of a set of constructive operators to a set of existing features resulting in construction of new features. For example, a machine learning system based on a neural network architecture may be provided image data from an image sensor. Early layers in the neural network may identify horizontal lines and vertical lines in the image data. Later layers in the network may then use the lines identified to obtain edges, a higher-level feature, for particles in the image.

A deep learning neural network may be a Multi Layer Perceptron (MLP), Convolutional Neural Network (CNN), or a Recurrent Neural Network (RNN).

### Assays to Obtain Genetic Data

The identification or analysis of one or more genetic modifiers of LRRK2 may include performing an assay on a sample obtained from a subject. The sample may be any type of sample that contains genetic material, such as DNA or RNA. For example and without limitation, the sample may be from an amniotic fluid, biopsy, blood, bodily fluid, cell, cerebrospinal fluid, lymphatic fluid, mouthwash, needle aspiration biopsy, hair, phlegm, plasma, pus, saliva, semen, serum, sputum, stool, swab, sweat, synovial fluid, tear, tissue, urine, or a combination of any of the aforementioned samples. For example and without limitation, a tissue sample may be from bone marrow tissue, CNS tissue, eye tissue, gastrointestinal tissue, genitourinary tissue, hair, kidney tissue, liver tissue, mammary gland tissue, mammary gland tissue, musculoskeletal tissue, nails, nasal passage tissue, neural tissue, placental tissue, placental tissue, or skin tissue.

The subject may be any type of subject. The subject may be a human. The subject may show one or more symptoms of Parkinson's disease, or the subject may be asymptomatic. The patient may be related to a PD patient. The subject may be a pediatric patient, a newborn, a neonate, an infant, a child, an adolescent, a pre-teen, a teenager, an adult, or an elderly subject. The subject may show one or more symptoms of Parkinson's disease, or the subject may be asymptomatic. The patient may be related to a PD patient.

Methods of genetic analysis are known in the art. In certain embodiments, a known single nucleotide polymorphism at a particular position can be detected by single base extension for a primer that binds to the sample DNA adjacent to that position, as described in, for example, U.S. Pat. No. 6,566,101, the content of which is incorporated by reference herein in its entirety. In other embodiments, a hybridization probe might be employed that overlaps the SNP of interest and selectively hybridizes to sample nucleic acids containing a particular nucleotide at that position, as described in, for example, U.S. Pat. Nos. 6,214,558 and 6,300,077, the contents of which are incorporated by reference herein in their entirety.

In particular embodiments, nucleic acids are sequenced in order to detect variants (i.e., mutations) in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art e.g., ensemble sequencing or single molecule sequencing.

Sequencing may be by any method known in the art. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes

One conventional method to perform sequencing is by chain termination and gel separation, as described in, for example, Sanger et al., Proc Natl. Acad. Sci. USA, 74(12): 5463 67 (1977). Another conventional sequencing method involves chemical degradation of nucleic acid fragments, as described in, for example, Maxam et al., Proc. Natl. Acad. Sci., 74: 560 564 (1977). Finally, methods have been developed based upon sequencing by hybridization, as described in, for example, U.S. Patent Publication number 2009/0156412. The content of each reference is incorporated by reference herein in its entirety.

A sequencing technique that can be used in the methods of the provided invention includes, for example, Harris T. D. et al., Single-Molecule DNA Sequencing of a Viral Genome, (2008) Science 320:106-109. In the true single molecule sequencing (tSMS) technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. The templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are detected by imaging the flow cell surface. After imaging, a cleavage step removes the fluorescent label, and the process is repeated with other fluorescently labeled nucleotides until the desired read length is achieved. Sequence information is collected with each nucleotide addition step. Further description of tSMS is shown for example in U.S. Pat. Nos. 7,169,560; 6,818,395; and 7,282,337; U.S. Patent Publication Nos. 2009/0191565 and 2002/0164629; and Braslavsky, et al., PNAS (USA), 100: 3960-3964 (2003), the contents of each of which are incorporated by reference herein in their entirety.

Another example of a DNA sequencing technique that can be used in the methods of the provided invention is 454 sequencing (Roche), as described in, for example, Margulies, M et al. 2005, Nature, 437, 376-380. 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is detected and analyzed.

Another example of a DNA sequencing technique that can be used in the methods of the provided invention is SOLiD technology (Applied Biosystems). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured, and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed, and the process is then repeated.

Another example of a DNA sequencing technique that can be used in the methods of the provided invention is Ion Torrent sequencing, as described in U.S. Patent Publication Nos. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, the contents of each of which are incorporated by reference herein in their entirety. In Ion Torrent sequencing, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to a surface and is attached at a resolution such that the fragments are individually resolvable. Addition of one or more nucleotides releases a proton (H⁺), which signal detected and recorded in a sequencing instrument. The signal strength is proportional to the number of nucleotides incorporated.

Another example of a sequencing technology that can be used in the methods of the provided invention is Illumina sequencing. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing, as described in, for example, Soni G V and Meller A. (2007) Clin Chem 53: 1996-2001. A nanopore is a small hole, of the order of 1 nanometer in diameter. Immersion of a nanopore in a conducting fluid and application of a potential across it results in a slight electrical current due to conduction of ions through the nanopore. The amount of current which flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore represents a reading of the DNA sequence.

Another example of a sequencing technique that can be used in the methods of the provided invention involves using a chemical-sensitive field effect transistor (chemFET) array to sequence DNA, for example, as described in U.S. Patent Publication No. 20090026082). In one example of the technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be detected by a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

Another example of a sequencing technique that can be used in the methods of the provided invention involves using an electron microscope, as described in, for example, Moudrianakis E. N. and Beer M. Proc Natl Acad Sci USA. 1965 March; 53:564-71. In one example of the technique, individual DNA molecules are labeled using metallic labels that are distinguishable using an electron microscope. These molecules are then stretched on a flat surface and imaged using an electron microscope to measure sequences.

If the nucleic acid from the sample is degraded or only a minimal amount of nucleic acid can be obtained from the sample, PCR can be performed on the nucleic acid in order to obtain a sufficient amount of nucleic acid for sequencing, as described in, for example, U.S. Pat. No. 4,683,195, the content of which is incorporated by reference herein in its entirety).

Methods of detecting levels of gene products (e.g., RNA or protein) are known in the art. Commonly used methods known in the art for the quantification of mRNA expression in a sample include northern blotting and in situ hybridization, as described in, for example, Parker & Barnes, Methods in Molecular Biology 106:247-283 (1999), the contents of which are incorporated by reference herein in their entirety; RNAse protection assays, Hod, Biotechniques 13:852 854 (1992), the contents of which are incorporated by reference herein in their entirety); and PCR-based methods, such as reverse transcription polymerase chain reaction (RT-PCR), Weis et al., Trends in Genetics 8:263 264 (1992), the contents of which are incorporated by reference herein in their entirety. Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in, for example, U.S. Patent Publication No. 2006/0195269, the content of which is hereby incorporated by reference in its entirety.

A differentially or abnormally expressed gene refers to a gene whose expression is activated to a higher or lower level in a subject suffering from a disorder, such as PD, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as PD, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10-fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure.

The first step is the isolation of mRNA from a target sample. The starting material is typically total RNA isolated from human tissues or fluids.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). The contents of each of these references are incorporated by reference herein in their entirety. In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE Complete DNA and RNA Purification Kit (EPICENTRE, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif, USA), or Lightcycler (Roche Molecular Biochemicals, Mannheim, Germany). In certain embodiments, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and actin, beta (ACTB). For performing analysis on pre-implantation embryos and oocytes, conserved helix-loop-helix ubiquitous kinase (CHUK) is a gene that is used for normalization.

A more recent variation of the RT-PCR technique is the real time quantitative PCR, which measures PCR product accumulation through a dual-labeled fluorogenic probe (i.e., TaqMan® probe). Real time PCR is compatible both with quantitative competitive PCR, in which internal competitor for each target sequence is used for normalization, and with quantitative comparative PCR using a normalization gene contained within the sample, or a housekeeping gene for RT-PCR. For further details see, e.g., Held et al., Genome Research 6:986 994 (1996), the contents of which are incorporated by reference herein in their entirety.

In another embodiment, a MassARRAY-based gene expression profiling method is used to measure gene expression. In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g., Ding and Cantor, Proc. Natl. Acad. Sci. USA 100:3059 3064 (2003).

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in U.S. Patent Publication No. 2006/0195269), the content of which is incorporated by reference herein in its entirety.

In a specific embodiment of the microarray technique, PCR amplified inserts of cDNA clones are applied to a substrate in a dense array, for example, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair-wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels, as described in, for example, Schena et al., Proc. Natl. Acad. Sci. USA 93(2):106 149 (1996), the contents of which are incorporated by reference herein in their entirety. Microarray analysis can be performed by commercially available equipment, following manufacturer's protocols, such as by using the Affymetrix GenChip technology, or Incyte's microarray technology.

Alternatively, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, levels of transcripts of marker genes in a number of tissue specimens may be characterized using a “tissue array” as described in, for example, Kononen et al., Nat. Med 4(7):844-7 (1998). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g., Velculescu et al., Science 270:484 487 (1995); and Velculescu et al., Cell 88:243 51 (1997), the contents of each of which are incorporated by reference herein in their entirety.

In other embodiments Massively Parallel Signature Sequencing (MPSS) is used to measure gene expression. This method, described by Brenner et al., Nature Biotechnology 18:630 634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Immunohistochemistry methods are also suitable for detecting the expression levels of the gene products of the present invention. Thus, antibodies (monoclonal or polyclonal) or antisera, such as polyclonal antisera, specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In certain embodiments, a proteomics approach is used to measure gene expression. A proteome refers to the totality of the proteins present in a sample (e.g., tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g., my mass spectrometry or N-terminal sequencing, and (3) analysis of the data using bioinformatics. Proteomics methods are valuable supplements to other methods of gene expression profiling, and can be used, alone or in combination with other methods, to detect the products of the diagnostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, for example, U.S. Pat. Nos. 6,925,389; 6,989,100; and 6,890,763, each of which is incorporated by reference herein in their entirety.

### Reports on Genetic Modifiers of LRRK2

Methods of the invention may include providing a report on the subject. The report may identify one or more genetic modifiers of LRRK2 in the genetic data from the subject. The report may contain additional information about the subject, such as age, sex, weight, height, genetic data, genomic data, or other health or medical information. The report may include other information related to PD. For example and without limitation, the report may contain information about symptoms of PD or genes associated with PD, such as the symptoms and genes described above.

The report may be provided in any suitable manner. For example and without limitation, the report may be provided on paper or on a display device, such as a computer monitor, telephone, portable electronic device, or the like.

The report may be provided to a healthcare provider, such as a physician or nurse. The report may provide the healthcare provider guidance on whether treatment of the subject with a LRRK2 inhibitor is appropriate. The report may provide the healthcare provider with instructions or recommendations for treating the subject with a LRRK2 inhibitor. The report may recommend that the healthcare provider prescribe or provide a LRRK2 inhibitor for the subject or otherwise instruct the subject to procure and take a LRRK2 inhibitor.

The report may include guidance on whether to use a second agent in addition to a LRRK2 inhibitor to treat the subject. The second agent may be a known therapeutic agent for treatment of PD, such as any of those described above.

### LRRK2 Inhibitors

Methods of the invention may include providing one or more LRRK2 inhibitors to a subject or recommending that a subject take one or more LRRK2 inhibitors. LRRK2 inhibitors are known in the art and described in, for example, International Patent Publication Nos. WO 2012/028629, WO 2012/058193, WO 2012/118679, WO 2012/143143, WO 2012/143144, WO 2014/001973, WO 2014/060112, WO 2014/060113, WO 2014/145909, WO 2014/160430, WO 2014/170248, WO 2015/092592, WO 2015/113451, WO 2015/113452, WO 2016/130920, WO 2017/012576, WO 2017/046675, WO 2017/087905, WO 2017/106771, WO 2017/156493, WO 2017/218843, WO 2018/137573, WO 2018/137593, WO 2018/137618, WO 2018/137619, WO 2018/163030, WO 2018/163066, WO 2018/217946, WO 2019/012093, WO 2019/104086, WO 2019/112269, WO 2019/126383, WO 2020/149723, WO 2020/170205, and WO 2020/210684; U.S. Pat. No. 9,499,535; co-pending U.S. Application Nos. 63/050,385, 63/133,523, 63/113,533, 63/137,814, 63/137816, and 63/142009; and co-pending International Application Nos. PCT/IB2020/000727, PCT/IB2020/000730, PCT/US2021/041270, and PCT/US2021/041271, the contents of each of which are incorporated herein by reference in their entirety. Any LRRK2 disclosed in any of the aforementioned references may be used in methods of the invention.

For example and without limitation, the LRRK2 inhibitor may be CZC-25146, CZC-54252, DNL151, DNL201, GNE-7915, GSK2578215A, HG-10-102-01, JH-II-127, K252A, K252B, LRRK2-IN-1, MLi-2, PF-06447475, or staurosporine.

In some methods of the invention, the LRRK2 inhibitor is a compound of one of formulas (I), (II), (III), and (IV).

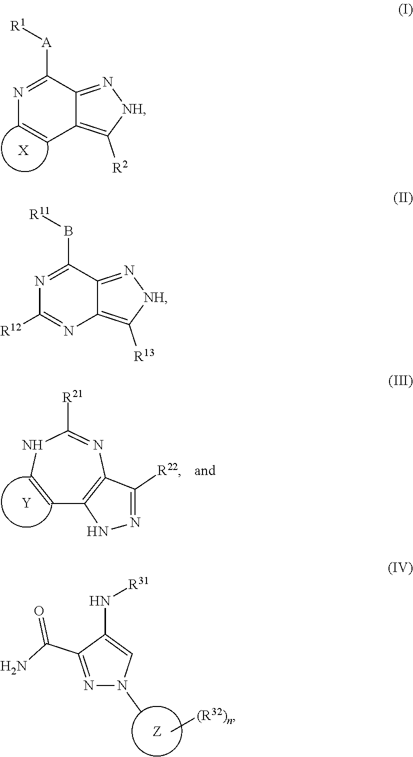

wherein:
A is NH, O, S, C═O, NR³ or CR⁴R⁵;
X is an optionally substituted arylene, heteroarylene, cycloalkylene, heterocycloalkylene, alkylcycloalkylene, heteroalkylcycloalkylene, aralkylene or heteroaralkylene group;
R¹ is an optionally substituted alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R² is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R⁴ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group; and
R^{S} is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
B is NH, O, S, C═O, NR¹⁴ or CR¹⁵R¹⁶;
R¹¹ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹² is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group, wherein R¹² is bound to the pyrimidine ring of formula (II) via a carbon-carbon bond;
R¹³ is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁴ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R¹⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁶ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R²¹ is aryl or heteroaryl, each of which is optionally substituted;
R²² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl; and
Y is aryl or 5- or 6-membered heteroaryl; wherein each of the C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, and heteroaryl is optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;
R³¹ is C(O)CH₂R³³, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R³² is independently halo, haloalkyl, optionally substituted alkoxyl, optionally substituted alkyl, optionally substituted heteroalkyl, optionally substituted alkenyl, optionally substituted heteroalkenyl;
R³³ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
Z is cycloalkyl, cycloheteroalkyl, cycloalkenyl, cycloheteroalkenyl, aryl, or heteroaryl; Z may be an aryl substituted with 2 or 3 instances of R₂. Z may be a phenyl substituted with 2 or 3 instances of R₂. Z may be a heteroaryl substituted with 2 or 3 instances of R₂. Z may be a six-membered heteroaryl substituted with 2 or 3 instances of R₂;
and
n is 0-5,
   or a pharmaceutically acceptable salt of any compound described above.

The LRRK2 inhibitor may be provided to a subject in a pharmaceutical composition. The pharmaceutical composition may contain the LRRK2 inhibitor in a therapeutically effective amount. A therapeutically effective amount means an amount that is effective to prevent, alleviate, or ameliorate symptoms of a disease, such as PD, or prolong the survival of the subject being treated. Determination of a therapeutically effective amount is within the skill in the art. The therapeutically effective amount or dosage of a LRRK2 inhibitor can vary within wide limits and may be determined in a manner known in the art. Such dosage may be adjusted to the individual requirements in each particular case including the specific compound being administered, the route of administration, the condition being treated, as well as the patient being treated.

For oral administration such therapeutically useful agents can be administered by one of the following routes: oral, e.g., as tablets, dragees, coated tablets, pills, semisolids, soft or hard capsules, for example, soft and hard gelatin capsules, aqueous or oily solutions, emulsions, suspensions or syrups, parenteral including intravenous, intramuscular and subcutaneous injection, e.g., as an injectable solution or suspension, rectal as suppositories, by inhalation or insufflation, e.g., as a powder formulation, as microcrystals or as a spray (e.g., liquid aerosol), transdermal, for example via an transdermal delivery system (TDS) such as a plaster containing the active ingredient or intranasal. For the production of such tablets, pills, semisolids, coated tablets, dragees and hard, e.g., gelatin, capsules, the therapeutically useful product may be mixed with pharmaceutically inert, inorganic or organic excipients as are e.g., lactose, sucrose, glucose, gelatin, malt, silica gel, starch or derivatives thereof, talc, stearinic acid or their salts, dried skim milk, and the like. For the production of soft capsules one may use excipients as are e.g., vegetable, petroleum, animal or synthetic oils, wax, fat, polyols. For the production of liquid solutions, emulsions or suspensions or syrups one may use as excipients e.g., water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g., vegetable, petroleum, animal or synthetic oils, wax, fat and polyols. For aerosol formulations one may use compressed gases suitable for this purpose, as are e.g., oxygen, nitrogen and carbon dioxide. The pharmaceutically useful agents may also contain additives for conservation, stabilization, e.g., UV stabilizers, emulsifiers, sweetener, aromatizers, salts to change the osmotic pressure, buffers, coating additives and antioxidants.

### Providing a LRRK2 Inhibitor to a Subject

Methods of the invention may include providing a LRRK2 inhibitor to a subject. The LRRK2 inhibitor may be provided by any suitable route or mode of administration. For example and without limitation, the compound may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device (e.g., stent or drug-eluting stent or balloon equivalents).

The LRRK2 inhibitor may be provided according to a dosing regimen. A dosing regimen may include a dosage, a dosing frequency, or both.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least one week, at least two weeks, at least three weeks, at least four weeks, at least six weeks, at least eight weeks, at least ten weeks, at least twelve weeks or more.

The subject may be any type of subject, such as any of those described above in relation to assays to obtain genetic data.

The invention includes combination therapies in which a LRRK2 inhibitor is provided to a subject in combination with a second agent, such as any of the drugs described above in the section on PD. The LRRK2 inhibitor and the second agent may be provided in a single composition, or they may be provided in separate compositions. The LRRK2 inhibitor and the second agent may be provided according to the same dosing regimen, or they may be provided according to different dosing regimens.

### EXAMPLES

### Example 1

Likelihood of responsiveness to LRRK2 inhibitors was analyzed in a population of human subjects. The datasets include the complete dataset from the Accelerating Medicines Partnership—Parkinson's Disease (AMP-PD). The input data was quality controlled data for Parkinson's disease cases focusing on clinical, demographic, RNA and DNA sequencing data at baseline in the available samples as of Jun. 1, 2020. Whole genome sequencing and RNA sequencing were processed using standard pipelines described on the AMP-PD website. Analyses were limited to samples with <15% data missiness rates after consensus quality control. Analyses were also rerun adjusting for inter-European population substructure yielding near identical results in the same set of >1000 cases.

To identify potential modifiers, the open-source automated machine learning package GenoML was used. This package carried out feature selection/weighting and normalization, then competed algorithms in randomly ascertained 70% training set and 30% test set. The best-performing algorithm in terms of balances accuracy was then selected for further tuning and cross validation. The best-performing algorithm was then hyperparameter-tuned using a randomized grid search method and 10-fold cross-validation was performed, with the focus of this tuning process was maximizing the balanced accuracy. The outcome was coded as 0/1, with a 1 being indicative of carrying a known LRRK2 causative variant. A matrix of probabilities for WT LRRK2 cases was exported, with these probabilities indicating how “LRRK2+ similar” they are on a molecular/clinical/demographic level. The most important features across all iterations of the model were used as potential regulatory factors.

Results are provided in Table 1.

TABLE 1

| SNP | P value | SNP | P value | SNP | P value | SNP | P value |
|---|---|---|---|---|---|---|---|
| rs10748409 | 5.68E−04 | rs149085137 | 1.51E−02 | rs201966701 | 4.36E−03 | rs710782041 | 3.35E−14 |
| rs10784722 | 5.42E−31 | rs149173058 | 4.74E−13 | rs201972304 | 9.42E−03 | rs71443939 | 2.84E−02 |
| rs10785367 | 1.47E−03 | rs149187646 | 1.01E−02 | rs201985363 | 1.46E−04 | rs7304080 | 1.92E−11 |
| rs10877367 | 2.97E−03 | rs149386982 | 3.45E−03 | rs202009839 | 2.79E−02 | rs73088926 | 1.64E−10 |
| rs10877877 | 2.41E−22 | rs149667443 | 3.45E−05 | rs202033705 | 3.46E−03 | rs73093306 | 2.69E−03 |
| rs10879122 | 8.54E−63 | rs150428618 | 2.65E−05 | rs202117834 | 1.06E−05 | rs73110320 | 3.45E−03 |
| rs10880213 | 3.25E−04 | rs150442003 | 3.45E−05 | — | n.a. | rs73122881 | 4.88E−02 |
| rs10880307 | 1.93E−05 | rs150564530 | 2.37E−05 | rs202163555 | 5.25E−03 | rs73278235 | 1.37E−02 |
| — | n.a. | rs166700 | 3.44E−02 | rs202215620 | 3.74E−09 | rs74075947 | 7.62E−04 |
| rs111498917 | 3.13E−06 | rs17092636 | 7.06E−03 | rs2134067 | 5.77E−05 | rs74434364 | 1.51E−68 |
| rs111620026 | 3.45E−05 | rs17558833 | 4.11E−03 | — | n.a. | — | n.a. |
| rs111703502 | 2.45E−03 | rs17580794 | 5.18E−07 | rs2406426 | 2.61E−07 | rs74662094 | 7.37E−03 |
| rs11180082 | 5.25E−03 | rs17621741 | 9.36E−13 | rs2406860 | 4.88E−12 | rs74842215 | 1.35E−13 |
| rs11181379 | 9.92E−08 | — | n.a. | rs2708049 | 2.86E−04 | — | n.a. |
| — | n.a. | rs17655662 | 1.71E−02 | — | n.a. | rs74940565 | 3.56E−06 |
| rs11181542 | 5.05E−15 | rs181074684 | 4.20E−04 | rs2731054 | 1.23E−05 | rs75043969 | 6.04E−14 |
| rs111824538 | 1.02E−02 | rs181125968 | 3.46E−03 | rs28406258 | 2.60E−08 | — | n.a. |
| rs11182654 | 1.94E−09 | rs181888197 | 3.34E−02 | rs285561 | 1.21E−10 | rs75295254 | 3.35E−06 |
| — | n.a. | rs182246725 | 1.02E−02 | rs28795105 | 1.59E−09 | rs75351256 | 5.46E−03 |
| rs11182666 | 2.14E−05 | rs182270031 | 2.77E−04 | — | n.a. | rs75559961 | 2.16E−02 |
| rs112093601 | 1.54E−04 | rs182597608 | 1.71E−02 | rs34063350 | 2.44E−04 | rs75562290 | 1.01E−02 |
| rs112246279 | 4.89E−02 | rs182732512 | 6.12E−04 | rs34566033 | 2.45E−10 | rs76092702 | 4.46E−05 |
| rs112525075 | 8.50E−03 | rs182938343 | 1.76E−04 | rs35020261 | 1.86E−03 | rs76264521 | 5.25E−03 |
| — | n.a. | rs182977052 | 7.90E−05 | rs35460323 | 3.79E−03 | rs76319690 | 1.13E−02 |
| rs112838640 | 8.13E−03 | rs183804981 | 2.18E−02 | rs367598356 | 4.84E−02 | rs76433878 | 2.41E−02 |
| rs113000149 | 1.58E−03 | rs183821466 | 3.45E−05 | rs367882436 | 4.31E−02 | rs76589567 | 4.69E−06 |
| rs113111234 | 2.24E−10 | rs1838354 | 5.89E−13 | rs368141132 | 1.44E−15 | rs76599951 | 9.09E−04 |
| rs113266646 | 3.45E−03 | — | n.a. | rs368339469 | 1.48E−02 | — | n.a. |
| rs113519186 | 7.29E−03 | rs184120094 | 8.08E−42 | rs368522121 | 2.12E−04 | rs76981669 | 3.45E−05 |
| rs113621899 | 6.69E−03 | rs184882646 | 1.46E−02 | — | n.a. | rs77072644 | 1.64E−02 |
| rs113736300 | 3.81E−08 | rs1849781 | 2.79E−02 | rs369084695 | 1.42E−10 | rs77405383 | 3.82E−02 |
| rs113894508 | 1.31E−03 | rs185075448 | 7.48E−06 | rs369288207 | 1.81E−06 | rs77502044 | 3.45E−03 |
| rs114908406 | 4.43E−02 | rs185879499 | 4.89E−02 | rs370061012 | 7.89E−03 | rs77608635 | 4.41E−02 |
| rs11520109 | 9.62E−06 | rs185945121 | 4.96E−03 | rs370356580 | 1.07E−02 | rs77714081 | 3.88E−02 |
| rs1160314 | 2.36E−02 | rs186449020 | 3.45E−03 | rs370900619 | 1.17E−03 | rs77878151 | 2.35E−03 |
| rs11615728 | 3.41E−04 | rs186529799 | 2.65E−05 | rs371478782 | 3.05E−08 | rs78321043 | 5.20E−04 |
| rs1168080 | 3.20E−02 | rs186704426 | 3.46E−03 | rs371594746 | 1.02E−02 | rs78323838 | 6.09E−03 |
| rs117129580 | 1.68E−03 | rs186827558 | 2.65E−05 | rs371700002 | 4.95E−13 | rs78450226 | 6.72E−06 |
| rs117189052 | 5.88E−03 | rs187183686 | 1.94E−02 | — | n.a. | — | n.a. |
| rs117269442 | 1.57E−03 | rs187348393 | 3.45E−03 | rs371764054 | 1.64E−02 | rs78468120 | 5.08E−37 |
| rs117852256 | 3.45E−03 | rs187374392 | 1.35E−05 | rs371870259 | 1.14E−05 | rs78468120 | 6.22E−03 |
| rs12230765 | 1.22E−21 | rs187782700 | 4.90E−04 | rs371905892 | 1.63E−14 | rs78936885 | 7.30E−07 |
| rs12297783 | 5.05E−03 | — | n.a. | — | n.a. | rs78936885 | 2.99E−02 |
| rs12305830 | 3.21E−02 | rs188073854 | 7.27E−04 | rs373439540 | 4.28E−16 | rs79021464 | 2.57E−02 |
| rs12309471 | 4.49E−02 | rs188203806 | 1.76E−04 | — | n.a. | rs7960429 | 4.54E−28 |
| rs12312256 | 3.59E−07 | rs188535877 | 2.80E−12 | rs374331320 | 8.92E−03 | rs7960950 | 1.03E−02 |
| rs12321815 | 3.35E−09 | rs188583486 | 9.92E−14 | rs375069269 | 4.13E−05 | rs7968196 | 1.11E−02 |
| — | n.a. | rs188604552 | 4.52E−19 | rs375125009 | 4.17E−04 | rs7969016 | 3.77E−06 |
| rs12369845 | 4.92E−02 | — | n.a. | rs375848639 | 2.17E−04 | rs7969372 | 4.31E−06 |
| rs12581873 | 2.77E−02 | rs188694711 | 4.31E−02 | rs376149390 | 4.27E−03 | rs7969614 | 5.91E−03 |
| rs12809884 | 3.45E−03 | rs189383941 | 1.57E−03 | rs376189144 | 2.48E−05 | — | n.a. |
| rs12812028 | 2.71E−03 | rs189517205 | 3.70E−15 | rs376468815 | 2.21E−20 | rs7979420 | 5.81E−15 |
| rs12816484 | 2.58E−10 | rs190706519 | 3.45E−03 | rs377104202 | 6.54E−15 | rs7980297 | 2.16E−02 |
| — | n.a. | rs190891557 | 4.09E−08 | rs377317160 | 2.75E−02 | rs79916386 | 4.69E−06 |
| rs12828885 | 1.81E−03 | rs191167563 | 4.16E−02 | rs377387769 | 1.64E−03 | rs80049049 | 1.90E−03 |
| — | n.a. | rs192166260 | 3.87E−02 | rs377458420 | 3.65E−06 | rs80089930 | 1.67E−02 |
| rs12829831 | 8.05E−15 | rs192242423 | 2.59E−03 | — | n.a. | rs824700 | 1.07E−04 |
| rs1283308 | 1.49E−09 | rs192943667 | 4.58E−02 | rs377627337 | 1.34E−11 | rs826874 | 2.80E−05 |
| rs13377670 | 2.83E−27 | rs199635668 | 1.57E−02 | rs377727699 | 1.07E−02 | rs9668004 | 3.45E−05 |
| rs138153837 | 1.71E−02 | rs199680813 | 4.43E−02 | rs384234 | 1.13E−26 | rs9783514 | 6.11E−06 |
| rs139014074 | 7.07E−03 | rs199845692 | 1.23E−02 | rs4087221 | 8.92E−03 | rs9795857 | 2.38E−07 |
| rs139191435 | 4.89E−02 | rs200090686 | 9.88E−04 | rs4251584 | 4.89E−02 | UPSIT | 7.8E−166 |
| rs139249809 | 3.19E−02 | rs200130007 | 4.16E−02 | rs4357757 | 1.28E−02 | — | n.a. |
| rs139547067 | 1.07E−02 | rs200211189 | 8.94E−04 | — | n.a. | — | n.a. |
| rs139602942 | 3.45E−03 | rs200300622 | 1.97E−04 | rs442215 | 1.58E−09 | — | n.a. |
| rs139985561 | 5.54E−03 | rs200462207 | 1.17E−02 | rs4491268 | 3.26E−04 | — | n.a. |
| rs140151156 | 4.31E−02 | rs200504831 | 4.52E−03 | rs4554929 | 3.65E−03 | — | n.a. |
| rs140252949 | 7.62E−04 | rs200512607 | 1.92E−03 | rs4768443 | 9.11E−05 | — | n.a. |
| rs140756694 | 7.37E−03 | rs200611801 | 6.28E−12 | rs4768596 | 9.19E−03 | — | n.a. |
| rs140770228 | 3.45E−03 | — | n.a. | rs4882836 | 3.45E−03 | — | n.a. |
| rs140788746 | 2.44E−04 | rs200625718 | 1.63E−04 | rs55891021 | 8.73E−03 | — | n.a. |
| rs141161737 | 5.01E−03 | rs200658955 | 4.30E−09 | rs57311265 | 1.69E−07 | — | n.a. |
| rs141376702 | 1.13E−02 | — | n.a. | rs57311265 | 7.29E−04 | — | n.a. |
| rs141551396 | 7.85E−13 | rs200685998 | 3.88E−02 | rs57370291 | 1.50E−03 | — | n.a. |
| rs141551396 | 3.91E−08 | rs200706295 | 5.59E−04 | rs57979417 | 2.71E−02 | — | n.a. |
| rs141886347 | 3.45E−03 | rs200809365 | 2.79E−02 | rs58401116 | 2.14E−04 | — | n.a. |
| rs141972270 | 7.78E−10 | rs200862192 | 8.03E−09 | rs59638216 | 3.26E−02 | — | n.a. |
| rs141972270 | 2.76E−02 | — | n.a. | rs59919856 | 2.71E−02 | — | n.a. |
| rs142535651 | 6.60E−04 | rs200880593 | 5.84E−03 | rs59969014 | 1.01E−07 | — | n.a. |
| rs143057260 | 3.45E−03 | rs200907772 | 1.11E−19 | rs60468343 | 7.66E−04 | — | n.a. |
| rs143233902 | 2.30E−02 | — | n.a. | rs60533585 | 5.95E−03 | — | n.a. |
| rs143556376 | 7.90E−03 | rs200914329 | 3.49E−07 | rs61754102 | 3.56E−05 | — | n.a. |
| rs143928408 | 3.76E−03 | rs200921556 | 4.10E−09 | rs61920963 | 3.45E−03 | — | n.a. |
| rs144176455 | 7.62E−04 | rs201006087 | 3.82E−02 | rs61920964 | 3.44E−16 | — | n.a. |
| rs144377852 | 3.16E−12 | rs201043857 | 4.90E−03 | rs61920964 | 7.73E−05 | — | n.a. |
| rs144377852 | 3.84E−04 | rs201058501 | 1.51E−04 | rs61920975 | 3.80E−07 | — | n.a. |
| rs144497252 | 4.89E−02 | rs201212549 | 1.33E−04 | rs61920975 | 2.79E−02 | — | n.a. |
| rs146200091 | 4.89E−02 | rs201294892 | 3.45E−03 | rs61924404 | 1.68E−04 | — | n.a. |
| rs146598979 | 3.45E−03 | rs201419373 | 3.45E−03 | rs61924459 | 5.74E−05 | — | n.a. |
| rs146815469 | 8.92E−03 | rs201447215 | 1.74E−04 | rs61933046 | 4.31E−02 | — | n.a. |
| rs1469567 | 1.76E−03 | rs201482136 | 5.29E−08 | rs61937837 | 1.05E−02 | — | n.a. |
| rs147222338 | 3.45E−03 | — | — | rs63465760 | 1.74E−03 | — | n.a. |
| rs147485833 | 1.94E−02 | rs201615583 | 3.45E−03 | rs63465760 | 1.07E−02 | — | n.a. |
| rs147745969 | 3.58E−02 | rs201727670 | 3.45E−03 | rs6581941 | 1.65E−17 | — | n.a. |
| rs147827874 | 1.19E−02 | rs201832867 | 8.92E−03 | rs6581941 | 1.13E−02 | — | n.a. |
| rs1478614 | 6.87E−07 | rs201889643 | 2.14E−11 | rs6582516 | 1.13E−04 | — | n.a. |
| rs148671186 | 4.78E−06 | — | — | rs6650226 | 4.67E−20 | — | n.a. |
| rs148749439 | 5.01E−03 | rs201909292 | 1.07E−02 | rs6650226 | 1.59E−02 | — | n.a. |
| rs148819144 | 2.14E−05 | rs201914843 | 4.18E−02 | rs68009273 | 3.98E−03 | — | n.a. |
| rs149016398 | 3.45E−03 | rs201944175 | 8.76E−20 | rs68144673 | 2.97E−06 | — | n.a. |
| rs57025360 | 3.74E−140 | rs3793397 | 0.008553538532 | rs147286120 | 0.08956736218 | — | n.a. |
| rs116912628 | 4.66E−95 | rs378690 | 0.0269476088 | rs13009437 | 0.09694772942 | — | n.a. |
| rs2404832 | 1.70E−66 | rs3729912 | 0.03767131611 | rs145801597 | 0.09979817995 | — | n.a. |
| rs4272849 | 7.52E−57 | rs75479531 | 0.04629773827 | rs187116363 | 0.1092242851 | — | n.a. |
| rs10877877 | 3.09E−54 | rs147286120 | 0.08956736218 | rs4414548 | 0.1198573595 | — | n.a. |
| rs116912628 | 3.25E−31 | rs13009437 | 0.09694772942 | rs1718880 | 0.1532681318 | — | n.a. |
| rs112515153 | 6.05E−30 | rs145801597 | 0.09979817995 | rs610037 | 0.1571391139 | — | n.a. |
| rs1838343 | 8.94E−21 | rs187116363 | 0.1092242851 | rs56045011 | 0.179251336 | — | n.a. |
| rs10784722 | 4.81E−20 | rs4414548 | 0.1198573595 | rs1991401 | 0.1924834797 | — | n.a. |
| rs10880342 | 2.93E−17 | rs1718880 | 0.1532681318 | rs6858566 | 0.201278931 | — | n.a. |
| rs11177660 | 9.77E−15 | rs610037 | 0.1571391139 | rs73188365 | 0.2066683723 | — | n.a. |
| rs4931640 | 1.53E−08 | rs56045011 | 0.179251336 | rs308303 | 0.3527537076 | — | n.a. |
| rs11052225 | 2.37E−06 | rs1991401 | 0.1924834797 | rs10790282 | 0.36458394 | — | n.a. |
| rs11052253 | 3.05E−06 | rs74390551 | 0.92570396 | rs79307177 | 0.3686424109 | — | n.a. |
| rs11584630 | 0.001105242031 | rs526507 | 0.9942087092 | rs183028452 | 0.4106129032 | — | n.a. |
| rs3793397 | 0.008553538532 | rs4326638 | 0.6677721937 | rs72907976 | 0.4274633444 | — | n.a. |
| rs378690 | 0.0269476088 | rs144665441 | 0.7303798634 | rs147755361 | 0.4696183565 | — | n.a. |
| rs3729912 | 0.03767131611 | rs111794893 | 0.7514817194 | rs117534366 | 0.4847172826 | — | n.a. |
| rs75479531 | 0.04629773827 | rs71609573 | 0.6117905692 | — | n.a. | — | — |

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method of treating a subject having Parkinson's disease associated with wild-type LRRK2, the method comprising:
providing a LRRK2 inhibitor to a subject that presents with Parkinson's disease and that has wild-type LRRK2 and a genetic modifier of wild type LRRK2 such that the subject will respond to the LRRK2 inhibitor, thereby treating Parkinson's disease associated with wild-type LRRK2 in the subject.

2. The method of claim 1, wherein the genetic data comprises sequence data.

3. The method of claim 2, wherein the genetic modifier comprises a single nucleotide polymorphism (SNP).

4. The method of claim 3, wherein the SNP is selected from the group consisting of rs10784722, rs10877877, rs10879122, rs11181542, rs113111234, rs113736300, rs12230765, rs12816484, rs12829831, rs13377670, rs141551396, rs144377852, rs149173058, rs17580794, rs17621741, rs1838354, rs184120094, rs188535877, rs188583486, rs188604552, rs189517205, rs200611801, rs200907772, rs201889643, rs201944175, rs2406426, rs2406860, rs285561, rs34566033, rs368141132, rs369084695, rs371700002, rs371905892, rs373439540, rs376468815, rs377104202, rs377627337, rs384234, rs61920964, rs6581941, rs6650226, rs71078241, rs7304080, rs73088926, rs74434364, rs74842215, rs75043969, rs78468120, rs7960429, rs7979420, rs76904798, rs57025360, rs112515153, rs10877877, rs10784722, rs4272849, rs2404832, rs117534366, rs1838343, rs10880342, rs11177660, rs183028452, rs116912628, rs147755361, rs11584630, rs3793397, rs111794893, rs4931640, rs526507, rs79307177, rs187116363, rs71609573, rs74390551, rs144665441, rs1718880, rs1991401, rs11052225, rs145801597, rs72907976, rs147286120, rs378690, rs73188365, rs610037, rs75479531, rs1112191556, rs308303, rs10790282, rs3729912, rs4326638, rs4414548, rs13009437, rs56045011, rs6858566, rs4425, and rs11052253.

5. The method of claim 1, wherein the LRRK2 inhibitor is selected from the group consisting of CZC-25146, CZC-54252, DNL151, DNL201, GNE-7915, GSK2578215A, HG-10-102-01, JH-II-127, K252A, K252B, LRRK2-IN-1, MLi-2, PF-06447475, and staurosporine.

6. The method of claim 1, wherein the LRRK2 inhibitor is a compound selected from the group consisting of formulas (I), (II), (III), and (IV): 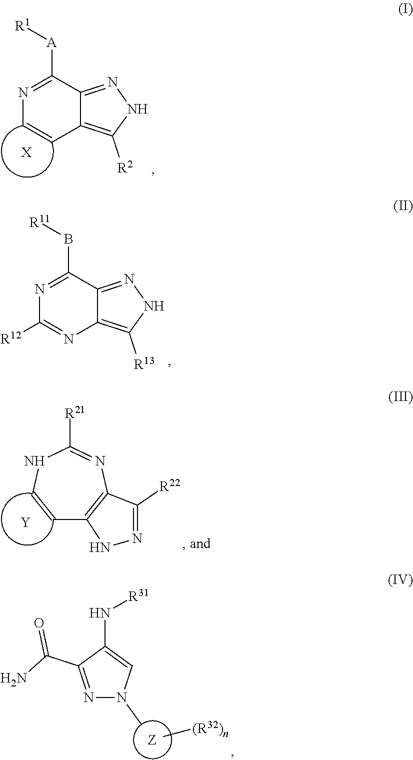
wherein:
A is NH, O, S, C═O, NR³ or CR⁴R⁵;
X is an optionally substituted arylene, heteroarylene, cycloalkylene, heterocycloalkylene, alkylcycloalkylene, heteroalkylcycloalkylene, aralkylene or heteroaralkylene group;
R¹ is an optionally substituted alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R² is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R⁴ is a hydrogen atom, NO₂, N₃, OH, SH, N₁₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group; and
R⁵ is a hydrogen atom, NO₂, N₃, OH, SH, N₁₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
B is NH, O, S, C═O, NR¹⁴ or CR¹⁵R¹⁶;
R¹¹ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹² is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group, wherein R¹² is bound to the pyrimidine ring of formula (II) via a carbon-carbon bond;
R¹³ is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁴ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R¹⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁶ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R²¹ is aryl or heteroaryl, each of which is optionally substituted;
R²² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl; and
Y is aryl or 5- or 6-membered heteroaryl; wherein each of the C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, and heteroaryl is optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;
R³¹ is C(O)CH₂R³³, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R³² is independently halo, haloalkyl, optionally substituted alkoxyl, optionally substituted alkyl, optionally substituted heteroalkyl, optionally substituted alkenyl, optionally substituted heteroalkenyl;
R³³ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
Z is cycloalkyl, cycloheteroalkyl, cycloalkenyl, cycloheteroalkenyl, aryl, or heteroaryl; Z may be an aryl substituted with 2 or 3 instances of R₂. Z may be a phenyl substituted with 2 or 3 instances of R₂. Z may be a heteroaryl substituted with 2 or 3 instances of R₂. Z may be a six-membered heteroaryl substituted with 2 or 3 instances of R₂; and
n is 0-5,
or a pharmaceutically acceptable salt of any compound described above.

7. A method of determining whether a subject having Parkinson's disease associated with wild-type LRRK2 will respond to a LRRK2 inhibitor, the method comprising:
conducting an assay on a sample from a subject that has Parkinson's disease associated with wild-type LRRK2 in order to obtain genetic data from the subject;
generating a report that identifies one or more genetic modifiers of LRRK2 in the genetic data, wherein the one or more genetic modifiers are indicative that the subject having Parkinson's disease associated with wild-type LRRK2 will be responsive to a LRRK2 inhibitor; and;
providing the report to a physician such that the physician prescribe or provide the subject with a LRRK2 inhibitor.

8. The method of claim 7, wherein the genetic data comprises sequence data.

9. The method of claim 8, wherein the genetic modifier comprises a single nucleotide polymorphism (SNP).

10. The method of claim 9, wherein the SNP is selected from the group consisting of rs10784722, rs10877877, rs10879122, rs11181542, rs113111234, rs113736300, rs12230765, rs12816484, rs12829831, rs13377670, rs141551396, rs144377852, rs149173058, rs17580794, rs17621741, rs1838354, rs184120094, rs188535877, rs188583486, rs188604552, rs189517205, rs200611801, rs200907772, rs201889643, rs201944175, rs2406426, rs2406860, rs285561, rs34566033, rs368141132, rs369084695, rs371700002, rs371905892, rs373439540, rs376468815, rs377104202, rs377627337, rs384234, rs61920964, rs6581941, rs6650226, rs71078241, rs7304080, rs73088926, rs74434364, rs74842215, rs75043969, rs78468120, rs7960429, rs7979420, rs76904798, rs57025360, rs112515153, rs10877877, rs10784722, rs4272849, rs2404832, rs117534366, rs1838343, rs10880342, rs11177660, rs183028452, rs116912628, rs147755361, rs11584630, rs3793397, rs111794893, rs4931640, rs526507, rs79307177, rs187116363, rs71609573, rs74390551, rs144665441, rs1718880, rs1991401, rs11052225, rs145801597, rs72907976, rs147286120, rs378690, rs73188365, rs610037, rs75479531, rs1112191556, rs308303, rs10790282, rs3729912, rs4326638, rs4414548, rs13009437, rs56045011, rs6858566, rs4425, and rs11052253.

11. The method of claim 7, wherein the method comprises identifying a plurality of genetic modifiers of LRRK2.

12. The method of claim 7, wherein the LRRK2 inhibitor is selected from the group consisting of CZC-25146, CZC-54252, DNL151, DNL201, GNE-7915, GSK2578215A, HG-10-102-01, JH-II-127, K252A, K252B, LRRK2-IN-1, MLi-2, PF-06447475, and staurosporine.

13. The method of claim 7, wherein the LRRK2 inhibitor is a compound selected from the group consisting of formulas (I), (II), (III), and (IV): 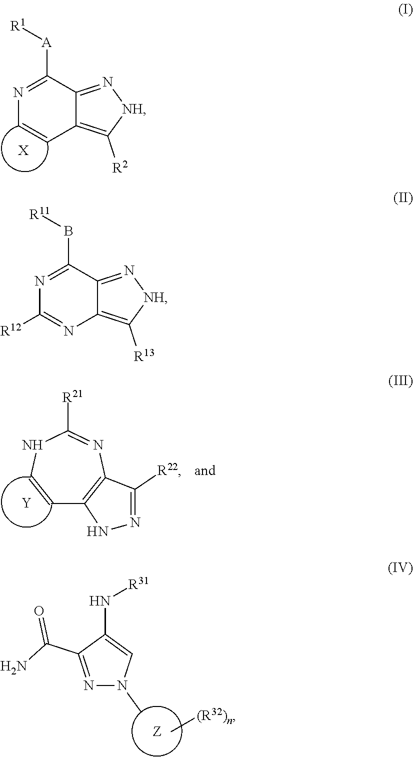
wherein:
A is NH, O, S, C═O, NR³ or CR⁴R⁵;
X is an optionally substituted arylene, heteroarylene, cycloalkylene, heterocycloalkylene, alkylcycloalkylene, heteroalkylcycloalkylene, aralkylene or heteroaralkylene group;
R¹ is an optionally substituted alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R² is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R⁴ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group; and
R⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
B is NH, O, S, C═O, NR¹⁴ or CR¹⁵R¹⁶;
R¹¹ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹² is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group, wherein R¹² is bound to the pyrimidine ring of formula (II) via a carbon-carbon bond;
R¹³ is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁴ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R¹⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁶ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R²¹ is aryl or heteroaryl, each of which is optionally substituted;
R²² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl; and
Y is aryl or 5- or 6-membered heteroaryl;
wherein each of the C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, and heteroaryl is optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;
R³¹ is C(O)CH₂R³³, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R³² is independently halo, haloalkyl, optionally substituted alkoxyl, optionally substituted alkyl, optionally substituted heteroalkyl, optionally substituted alkenyl, optionally substituted heteroalkenyl;
R³³ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
Z is cycloalkyl, cycloheteroalkyl, cycloalkenyl, cycloheteroalkenyl, aryl, or heteroaryl; and
n is 0-5,
or a pharmaceutically acceptable salt of any compound described above.

14. A method of treating a subject having Parkinson's disease associated with wild-type LRRK2, the method comprising:
receiving genetic data that identifies one or more genetic modifier of LRRK2, wherein the one or more genetic modifiers are indicative that a subject having Parkinson's disease associated with wild-type LRRK2 will be responsive to a LRRK2 inhibitor;
prescribing or providing the subject with a LRRK2 inhibitor.

15. (canceled)

16. The method of claim 14, wherein the genetic data comprises sequence data.

17. The method of claim 16, wherein the genetic modifier comprises a single nucleotide polymorphism (SNP).

18. The method of claim 17, wherein the SNP is selected from the group consisting of rs10784722, rs10877877, rs10879122, rs11181542, rs113111234, rs113736300, rs12230765, rs12816484, rs12829831, rs13377670, rs141551396, rs144377852, rs149173058, rs17580794, rs17621741, rs1838354, rs184120094, rs188535877, rs188583486, rs188604552, rs189517205, rs200611801, rs200907772, rs201889643, rs201944175, rs2406426, rs2406860, rs285561, rs34566033, rs368141132, rs369084695, rs371700002, rs371905892, rs373439540, rs376468815, rs377104202, rs377627337, rs384234, rs61920964, rs6581941, rs6650226, rs71078241, rs7304080, rs73088926, rs74434364, rs74842215, rs75043969, rs78468120, rs7960429, rs7979420, rs76904798, rs57025360, rs112515153, rs10877877, rs10784722, rs4272849, rs2404832, rs117534366, rs1838343, rs10880342, rs11177660, rs183028452, rs116912628, rs147755361, rs11584630, rs3793397, rs111794893, rs4931640, rs526507, rs79307177, rs187116363, rs71609573, rs74390551, rs144665441, rs1718880, rs1991401, rs11052225, rs145801597, rs72907976, rs147286120, rs378690, rs73188365, rs610037, rs75479531, rs1112191556, rs308303, rs10790282, rs3729912, rs4326638, rs4414548, rs13009437, rs56045011, rs6858566, rs4425, and rs11052253.

19. The method of claim 14, wherein the method comprises identifying a plurality of genetic modifiers of LRRK2.

20. The method of claim 14, wherein the LRRK2 inhibitor is selected from the group consisting of CZC-25146, CZC-54252, DNL151, DNL201, GNE-7915, GSK2578215A, HG-10-102-01, JH-II-127, K252A, K252B, LRRK2-IN-1, MLi-2, PF-06447475, and staurosporine.

21. The method of claim 14, wherein the LRRK2 inhibitor is a compound selected from the group consisting and formulas (I), (II), (III), or (IV): 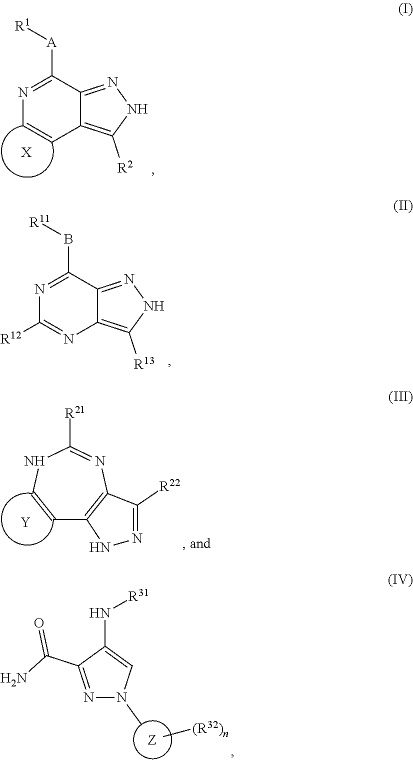
wherein:
A is NH, O, S, C═O, NR³ or CR⁴R⁵;
X is an optionally substituted arylene, heteroarylene, cycloalkylene, heterocycloalkylene, alkylcycloalkylene, heteroalkylcycloalkylene, aralkylene or heteroaralkylene group;
R¹ is an optionally substituted alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R² is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R⁴ is a hydrogen atom, NO₂, N₃, OH, SH, N₁₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group; and
R⁵ is a hydrogen atom, NO₂, N₃, OH, SH, N₁₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
B is NH, O, S, C═O, NR¹⁴ or CR¹⁵R¹⁶;
R¹¹ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹² is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group, wherein R¹² is bound to the pyrimidine ring of formula (II) via a carbon-carbon bond;
R¹³ is a hydrogen atom, a halogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁴ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl group;
R¹⁵ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R¹⁶ is a hydrogen atom, NO₂, N₃, OH, SH, NH₂ or an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl group;
R²¹ is aryl or heteroaryl, each of which is optionally substituted;
R²² is H, halo, OH, CN, CF₃, C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, or heteroaryl; and
Y is aryl or 5- or 6-membered heteroaryl;
wherein each of the C₁₋₆ alkyl, C₁₋₆ alkoxy, C₁₋₆ haloalkyl, C₁₋₆ thioalkyl, C₃₋₈ cycloalkyl, C₂₋₈ heterocycloalkyl, aryl, and heteroaryl is optionally substituted with one or more moieties selected from the group consisting of halo, OH, CN, CF₃, NH₂, NO₂, C₁₋₆ alkyl, C₁₋₆ haloalkyl, C₁₋₆ thioalkyl, C₃₋₈ cycloalkyl, C₂₋₈ heterocycloalkyl, C₂₋₈ heterocycloalkenyl, C₂₋₆ alkenyl, C₂₋₆ alkynyl, C₁₋₆ alkoxy, C₁₋₆ haloalkoxy, C₁₋₆ alkylamino, C₂₋₆ dialkylamino, C₇₋₁₂ aralkyl, C₁₋₁₂ heteroaralkyl, aryl, heteroaryl, —C(O)R, —C(O)OR, —C(O)NRR′, —C(O)NRS(O)₂R′, —C(O)NRS(O)₂NR′R″, —OR, —OC(O)NRR′, —NRR′, —NRC(O)R′, —NRC(O)NR′R″, —NRS(O)₂R′, —NRS(O)₂NR′R″, —S(O)₂R, and —S(O)₂NRR′,
in which each of R, R′, and R″, independently, is H, halo, OH, C₁₋₆ alkyl, C₁₋₆ haloalkyl, C₁₋₆ alkoxy, C₃₋₈ cycloalkyl, C₂₋₈ heterocycloalkyl, aryl, or heteroaryl, or R and R′, or R′ and R″, together with the nitrogen to which they are attached, form C₂₋₈ heterocycloalkyl;
R³¹ is C(O)CH₂R³³, optionally substituted cycloalkyl, optionally substituted cycloheteroalkyl, optionally substituted cycloalkenyl, optionally substituted cycloheteroalkenyl, optionally substituted aryl, or optionally substituted heteroaryl;
each instance of R³² is independently halo, haloalkyl, optionally substituted alkoxyl, optionally substituted alkyl, optionally substituted heteroalkyl, optionally substituted alkenyl, optionally substituted heteroalkenyl;
R³³ is optionally substituted cycloalkyl, optionally substituted cycloheteroalkyl, optionally substituted cycloalkenyl, optionally substituted cycloheteroalkenyl, optionally substituted aryl, or optionally substituted heteroaryl;
Z is cycloalkyl, cycloheteroalkyl, cycloalkenyl, cycloheteroalkenyl, aryl, or heteroaryl; and
n is 0-5,
or a pharmaceutically acceptable salt of any compound described above.
